(19) United States
(12) Patent Application Publication
(10) Pub. No.: US 2022/0354552 A1
(43) Pub. Date: Nov. 10, 2022

(54) SYSTEM AND METHOD FOR BONE FIXATION

(71) Applicant: NEXTREMITY SOLUTIONS, INC., Warsaw, IN (US)

(72) Inventors: Lon S. WEINER, Warsaw, IN (US); Stuart D. KATCHIS, Warsaw, IN (US); John R. PEPPER, Warsaw, IN (US); Ryan SCHLOTTERBACK, Warsaw, IN (US)

(73) Assignee: NEXTREMITY SOLUTIONS, INC., Warsaw, IN (US)

(21) Appl. No.: 17/662,557

(22) Filed: May 9, 2022

Related U.S. Application Data
(60) Provisional application No. 63/185,761, filed on May 7, 2021.
(60) Provisional application No. 63/244,310, filed on Sep. 15, 2021.

(51) Int. Cl. A61B 17/80; A61B 17/88; A61B 17/86; A61B 17/84

(52) U.S. Cl. CPC A61B 17/8057; A61B 17/8061; A61B 17/8875; A61B 17/8685; A61B 17/842

(57) ABSTRACT

A fracture fixation system includes a connector having a proximal connecting end for connecting to a first end of a cord configured to extend around a bone to provide fixation to a fracture of the bone. The connector includes a distal connecting end opposite the connector relative to the proximal end for attaching to a second end of the cord extending around the bone. The proximal connecting end includes a proximal lock member, a distal lock member and a medial lock member between the proximal lock member and the distal lock member. The proximal lock member is located at a bottom of the proximal connecting end and the medial lock member is located at a top of the proximal connecting end.

### CROSS REFERENCE TO RELATED APPLICATIONS

The present application claims priority to U.S. Provisional Application No. 63/185,761 filed May 7, 2021 (Attorney Docket No. 3768.098P3) and U.S. Provisional Application No. 63/244,310 filed Sep. 15, 2021 (Attorney Docket No. 3768.098P4).

The present application is related to U.S. Provisional Application No. 63/244,310 filed Sep. 15, 2021 (Attorney Docket No. 3768.098P4), U.S. Provisional Application No. 63/115,460 filed Nov. 18, 2020 (Attorney Docket No. 3768.098P2), U.S. Provisional Application No. 63/043,841 filed Jun. 25, 2020 (Attorney Docket No. 3768.098P1), U.S. application Ser. No. 16/910,328 filed Jun. 24, 2020 (Attorney Docket No. 3768.085A), U.S. application Ser. No. 17/358,706 filed Jun. 25, 2021 (Attorney Docket No. 3768.098A), and U.S. application Ser. No. 17/455,049 filed Nov. 16, 2021 (Attorney Docket No. 3768.098A2), all of which are incorporated herein by referenced in their entirety.

### FIELD OF THE INVENTION

This application relates generally to apparatuses, devices, and methods for bone fixation and more particularly to apparatuses, devices, and methods for cerclage related to skeletal fracture fixation and instrumentation to facilitate fracture reduction.

### BACKGROUND

Femoral fractures may occur naturally or iatrogenically during total hip arthroplasty. Depending on the fracture pattern, cerclage alone, cerclage with a plate or plates, cerclage with a strut, cerclage with an extended hip stem, or cerclage with a combination of plates, struts, and/or extended hip stems may be used for bone fixation. However, a problem with cerclage is that it may create poor bone unions without using additional support devices. In particular with total hip arthroplasty using additional support may not be desirable or possible. With femoral fractures, there may be a high load on the cerclage wires resulting in adjacent boney erosion and loss of fixation due to wire migration.

Fracture reduction of long bones often involves realigning spiral fractures and multiple pieces. Bone clamps are often hinged devices (e.g., pliers) with specialized tips or spurs to grab (e.g., engage and hold) bone. The closing of such a clamp puts force on the fragments and pushes them closer together. Limits of traditional clamps are they produce forces linearly, or very close to linearly. Such clamps may also be bulky when multiple clamps are used, as is common, access to a fracture site for placement of hardware (e.g., plates or screws) may be is restricted. Also, single plane forces produced by a clamp cannot adequately reduce some fractures. Further, clamps must be removed after fixation, and sometime loss of reduction and malalignment may occur due to such removal.

In another example, solid cerclage wire has been used to provide circumferential forces to reduce fractures. Use of such wire has some drawbacks, including the wire may be so stiff that manipulating the wire may be very difficult. Also, if the wire is not initially placed ideally, reforming the wire to fit in another location is nearly impossible. Further, the traditional method of tensioning solid cerclage wire involves twisting the ends in a tight spiral. This required specialized bulky tools, and the resulting twisted wire may be extremely stiff and often cannot be flattened to either work around. If the twisted wire is left in place, the wire may irritate soft tissues. These limitations have seen the use of solid temporary wire fixation drop to such that this method is rarely used.

There is a need for a device that provides improved bone unions while minimizing boney erosion and loss of fixation due to wire migration.

### SUMMARY OF THE INVENTION

The present invention provides, in a first aspect, a fracture fixation system which includes a connector having a proximal connecting end for connecting to a first end of a cord configured to extend around a bone to provide fixation to a fracture of the bone. The connector includes a distal connecting end opposite the connector relative to the proximal end for attaching to a second end of the cord extending around the bone. The proximal connecting end includes a proximal lock member, a distal lock member and a medial lock member between the proximal lock member and the distal lock member. The proximal lock member is located at a bottom of the proximal connecting end and the medial lock member is located at a top of the proximal connecting end.

The present invention provides, in a second aspect, a method for fixating a bone which includes aligning opposing portions of a fractured bone to set the bone and attaching a first end of a cord to a distal connecting end of a connector. A second end of the cord is attached to a proximal connecting end of the connector by threading the cord through a plurality of slots bounded by a proximal lock member, a distal lock member and a medial lock member between the proximal lock member and the distal lock member. The proximal lock member is located at the bottom of the proximal connecting end and the medial lock member is located at the top of the proximal connecting end. A force is applied to pull the cord around and through the proximal lock member, the distal lock member, and the medial lock member such that the cord is held at a particular tension to fixate the bone.

### BRIEF DESCRIPTION OF THE DRAWINGS

The present invention will be understood more fully from the detailed description given hereinafter and from the accompanying drawings of the preferred embodiment of the present invention, which, however, should not be taken to limit the invention, but are for explanation and understanding only.

FIG. 1 is a perspective view of a system for fixing a bone including a buckle and cord in;

FIG. 2 is a cross-sectional view of a portion of the buckle of FIG. 1 including locking bars;

FIG. 3 is a side cross-sectional view of the buckle of FIG. 1 including locking bars and showing a path of the cord of FIG. 1 around the locking bars;

FIG. 4 is a side view of a distal end of the cord of FIG. 1 including a loop for attaching to the buckle;

FIG. 5 is a side view of a passer including the cord of FIG. 1 received in a cavity therein;

FIG. 6 is a side view of the passer of FIG. 5;

FIG. 7 is a side view of the system of FIG. 5 including the cord attached to a proximal end of the buckle and a loop of the cord configured to be attached to the buckle;

FIG. 8 is a side view of the system of FIG. 7 with the buckle and loop attached to each other;

FIG. 9 is a side view of the system of FIG. 8 further including a tensioner;

FIG. 10 is a side view of the system of FIG. 9 with the proximal end of the cord received through a passage of the tensioner;

FIG. 11 is a side view of the system of FIG. 10 with the tensioner aligned to connect to the buckle;

FIG. 12 is a side view of the system of FIG. 11 showing the direction of rotation of a handle of the tensioner to draw the cord to tighten the cord relative to the buckle and the bone;

FIG. 13 is a side view of the system of FIG. 11 showing the cord being cut;

FIG. 14 is a perspective view of the tensioner of FIG. 13;

FIG. 15 is a perspective view of another example of a buckle useful in place of the buckle of FIG. 1;

FIG. 16 is a top view of the buckle of FIG. 15;

FIG. 17 is a perspective view of a plate having a plurality of buckles in accordance with the present invention;

FIG. 18 is a top view of the plate of FIG. 17;

FIG. 19 is a perspective view of a washer having a plurality of lock bars in accordance with the present invention.

FIG. 20 is a perspective view of a plate for use in fixating a bone in accordance with the present invention;

FIG. 21 is a close-up view of the plate of FIG. 20;

FIG. 22 is a perspective view of the plate of FIG. 20;

FIG. 23 is a close-up view of a portion of the plate of FIG. 20, including anti-migration projection;

FIG. 24 is a side view of a system for fixating a bone utilizing the plate of FIG. 20;

FIG. 25 is a close-up view of the system of FIG. 24, including screws through the plate of FIG. 20;

FIG. 26 is a perspective view of a tensioner engaged with a buckle and a bone to be fixated;

FIG. 27 is a side cross-sectional view of the tensioner of FIG. 26;

FIG. 28 is a close-up of a portion of the tensioner of FIG. 27;

FIG. 29 is a side view of a portion of the cross-sectional view of FIG. 28;

FIG. 30 is an exploded view of the tensioner of FIG. 26;

FIG. 31 is a cross-sectional view of the exploded view of the tensioner of FIG. 30;

FIG. 32 is a cross-sectional view of the tensioner of FIG. 27 at an oblique angle relative to an axis of the tensioner showing a lock for arms of the tensioner in a locked position;

FIG. 33 is a cross-sectional view as in FIG. 32 with the lock in a non-engaged position;

FIG. 34 is a perspective view of a tightener of the tensioner of FIG. 30;

FIG. 35 is a cross-sectional perspective view of the tightener of FIG. 34;

FIG. 36 is a perspective view of a puller of the tensioner of FIG. 30 exploded to show a connection of a cord to the puller;

FIG. 37 depicts an end view of the puller of FIG. 36;

FIG. 38 is a perspective end view of the puller of FIG. 37;

FIG. 39 is a top view of the cord of FIG. 36;

FIG. 40 is a perspective view of a modular passer having a handle connected to an arm and a second arm depicted for releasable attachment to the handle;

FIG. 41 is a perspective view of the arm from FIG. 40 shown separated from the handle, by itself;

FIG. 42 is a rear perspective view of the arm of FIG. 41;

FIG. 43 is a front perspective view of the passer and attached handle from FIG. 40;

FIG. 44 is a front perspective view of the passer and handle from FIG. 43 with a Locking switch in a release position;

FIG. 45 is a perspective view of a portion of the arm of FIG. 44 showing a hole for receiving a cord;

FIG. 46 is a cross section of the arm of FIG. 45;

FIG. 47 is a perspective view of a connector received on a bone plate with a cord extending from the connector around a bone to a second bone plate and returning to the connector;

FIG. 48 is a prospective view of a tensioner engaging the connect your end cord of FIG. 47 two tighten the cord;

FIG. 49 is a perspective view of a connector received on a bone plate with a cord extending from the connector around a bone and returning to the connector;

FIG. 50 is a perspective view of an example of a connector or buckle;

FIG. 51 is a cross sectional view of the connector of FIG. 50; and

FIG. 52 is a side cross section view of the connector of 51 showing a path of a cord through the lock members of the connector.

### DETAILED DESCRIPTION OF THE EMBODIMENTS

The present invention will be discussed hereinafter in detail in terms of various exemplary embodiments according to the present invention with reference to the accompanying drawings. In the following detailed description, numerous specific details are set forth in order to provide a thorough understanding of the present invention. It will be obvious, however, to those skilled in the art that the present invention may be practiced without these specific details. In other instances, well-known structures are not shown in detail in order to avoid unnecessary obscuring of the present invention. Thus, all the implementations described below are exemplary implementations provided to enable persons skilled in the art to make or use the embodiments of the disclosure and are not intended to limit the scope of the disclosure, which is defined by the claims.

The following description references systems, methods, and apparatuses for use in femoral fixation. However, those possessing an ordinary level of skill in the relevant art will appreciate that fixation of other bones are suitable for use with the foregoing systems, methods and apparatuses. Likewise, the various figures, steps, procedures and work-flows are presented only as an example and in no way limit the systems, methods or apparatuses described to performing their respective tasks or outcomes in different time-frames or orders. The teachings of the present invention may be applied to fixation related to any bone.

Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

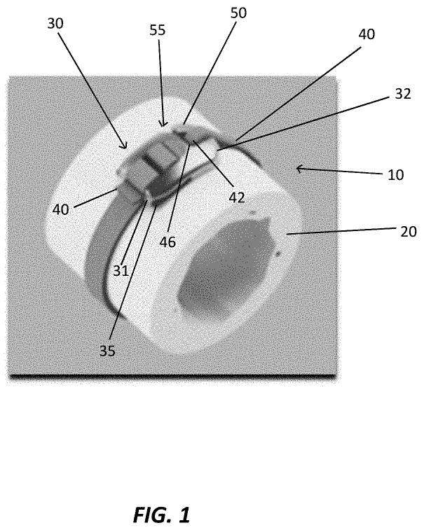

A system 10 for fixating or reducing a bone 20 may include a connector or buckle 30 connected to a strap, belt or cord 40 extending around bone 20, as depicted in FIG. 1.

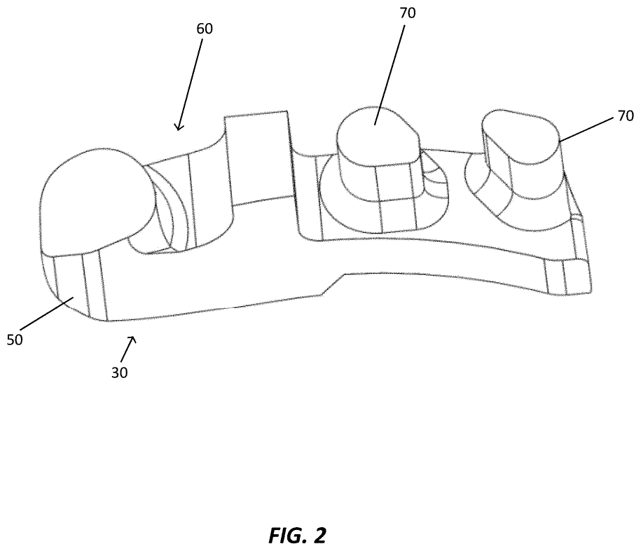

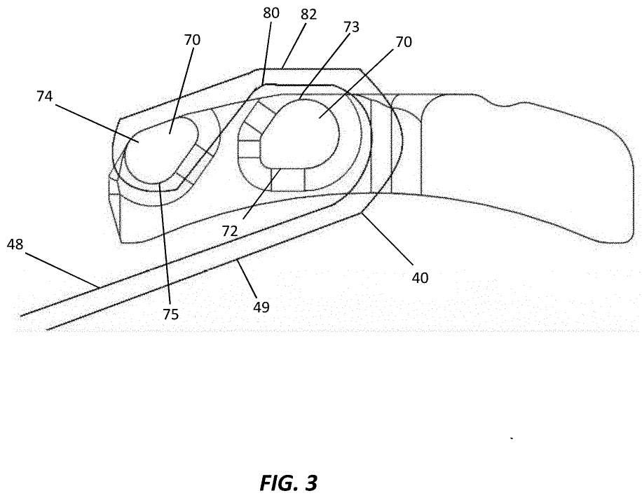

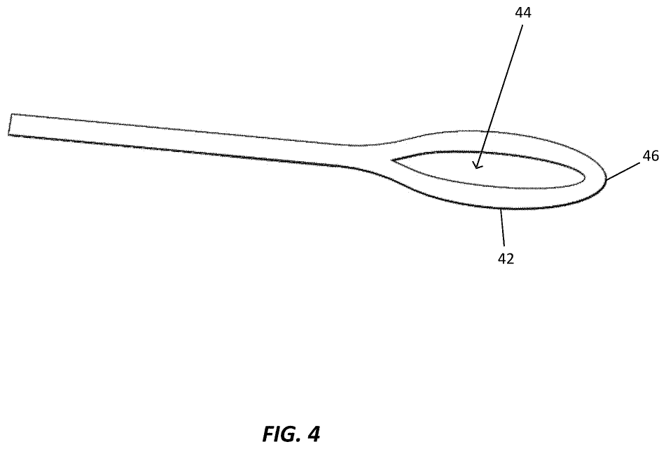

Buckle 30 may include a plurality of lock bars 70 and a hook 50 as depicted cross-sectionally in FIGS. 2-3. Lock bars may have a cross-sectional tear drop shape as depicted in the figures or could have a cross-sectional rounded shape. Hook 50 may be located at a distal end of buckle 30 and may have an opening 55 to allow a cord (e.g., cord 40) to pass therethrough into a receiving cavity 60. Cord 40 may have a loop 42 connectable to a distal portion 32 of buckle 30 when an opening 44 of loop 42 receives distal portion therein. When connected to buckle 30, a distal end 46 of cord 40 may be received in cavity 60. Cord 40 may also be connected to buckle 30 in other ways besides a loop, such as a knot, a ferrule, a non-eye splice, hitch or embedded internal stopper.

Buckle 30 could be made of stainless steel, surgical grade plastic, Titanium, PEEK, or Cobalt Chrome, for example. Also, cord 40 may be made of a material that may be left in a body for a period of time to allow a bone to heal, such as an Ultra-High Molecular Weight Polyethylene (UHMWPE), for example. Cord 40 may be a 50 cm tape with a width of 3.5 mm and a thickness of 0.6 mm, for example. Further cord 40 may be flexible (e.g., a suture tape) and may be made from strands, or braided stranded, of fine wires of metal or metallic alloy, such as cobalt chrome, stainless steel, titanium and titanium alloys.

A proximal end 48 of cord 40 may be connected to a proximal end 31 of buckle 30. For example, cord 40 may be connected to lock bars 70 as depicted in FIGS. 2-3. After extending around bone 20 as depicted in FIG. 1, cord 40 may extend distally toward hook 50 under lock bars 70 and extend upwardly away from bone 20, before reversing course and turning proximally away from hook 50 around a distal bar 72 of locking bars 70. Cord 40 may extend from distal bar 72 proximally away from hook 50 and then downwardly toward bone 20 before extending distally such that cord 40 goes around a proximal bar 74 of lock bars 70. From a bottom side 75 of proximal bar 74, cord 40 may extend upwardly and distally toward a top side 73 of distal bar 72. Cord 40 may extend upwardly over and/or contact top side 73 with a first cord portion 80 thereof while a second cord portion 82 may contact an opposite side of first cord portion 80 relative to top side 73. Cord 40 may extend from top side 73 distally toward hook 50 then downwardly toward bone 20 while contacting bar 72. First cord portion 80 and second cord portion may contact each other as cord 40 extends proximally from bar 72 away from hook 50. Proximal end 48 of cord 40 may be located on top of a distally extending portion 49 extending away from locking bars 70 around bone 20 toward hook 20. Proximal end 48 may be pulled by a user in a direction opposite from hook 50 to pull cord 40 through and past locking bars 70 as described above. A path of cord 40 past and against bars 70 may provide a frictional resistance to a release of cord 40 from bars 70 such that cord 40 may be tightened and such position retained to hold fractured portions of bone 20 for a period of time, or may remain in place, to allow a reduction of a fracture.

As described, system 10 may be utilized for fixating or reducing bone 20 and a process for such fixating or reducing is described as follows.

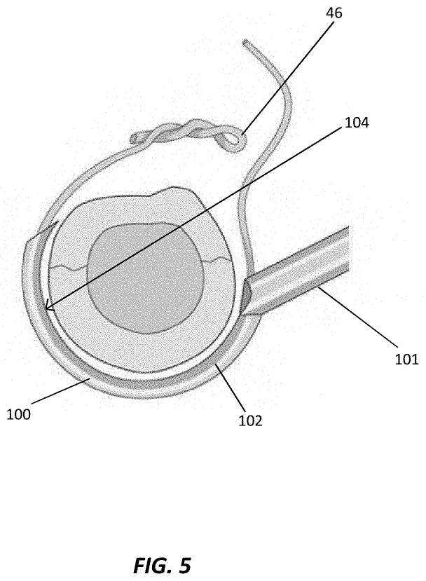

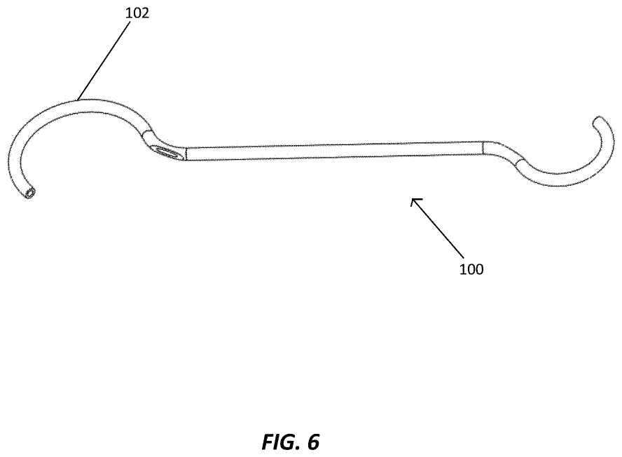

Cord 40 may be passed around bone 20 and under tissue and muscle 11 using a passer 100 as depicted in FIGS. 5-6. Passer 100 may have a curved extension portion 102 having a cavity 104 along an inner circumferential side for receiving cord 40 prior to a procedure such that passer 100 holding cord 40 in cavity 104 may be manipulated around bone 20 and under tissue and muscle 11, such that distal end 46 may be passed to an opposite side of bone 20 relative to a handle portion 101 of passer 100. Passer 100 may be removed from bone 20 with cord 40 remaining around bone 20 such that distal end 46 and proximal end 48 may be located on a same side of bone 20.

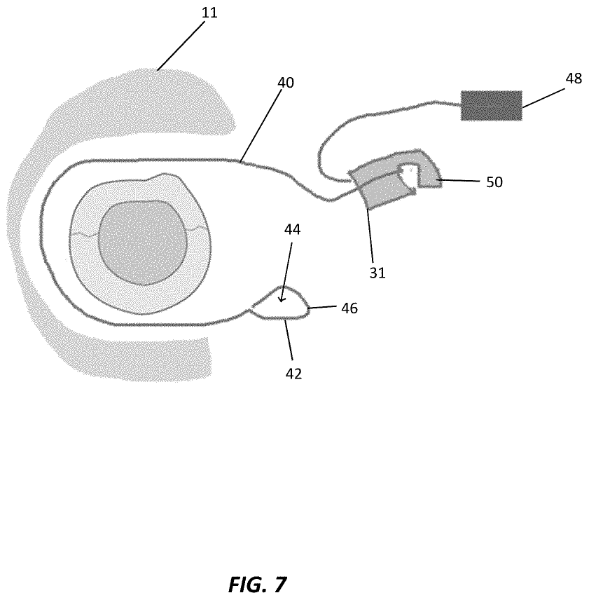

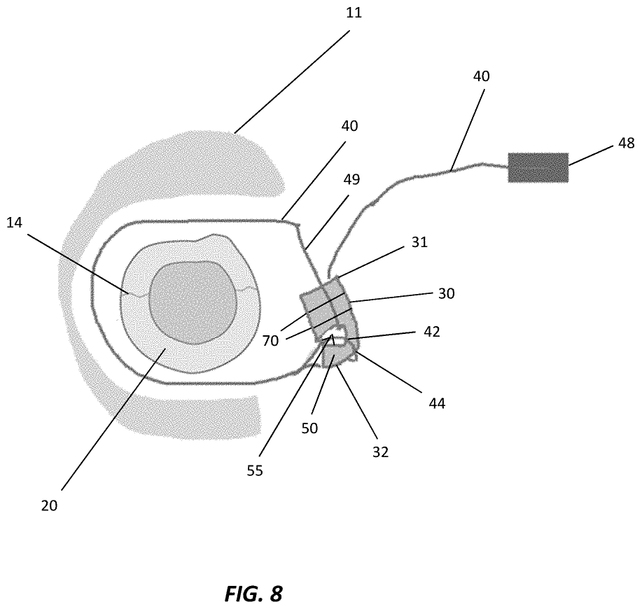

As depicted schematically in FIGS. 7-9, loop 44 of cord 40 may be connected to hook 50 and received in cavity 55. Cord 40 may be threaded around and connected to lock bars 70 as described above, depicted in FIGS. 1-3, and depicted schematically in FIGS. 7-9. A user may tighten cord 40 around bone 20 to reduce a fracture 14 of bone 20 by holding buckle 30 while pulling proximal end 48.

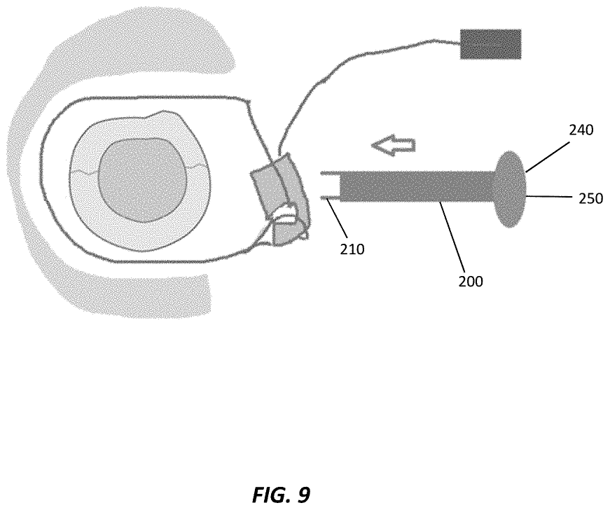

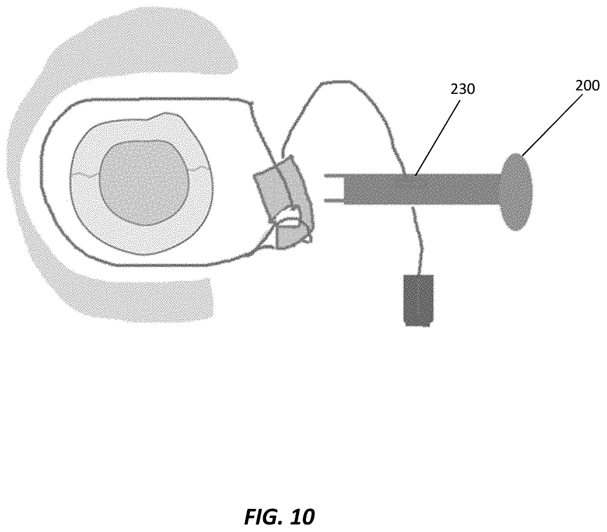

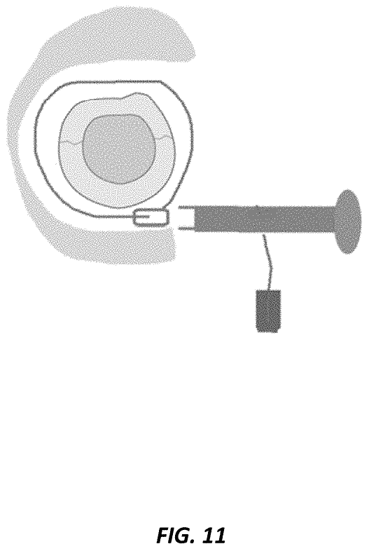

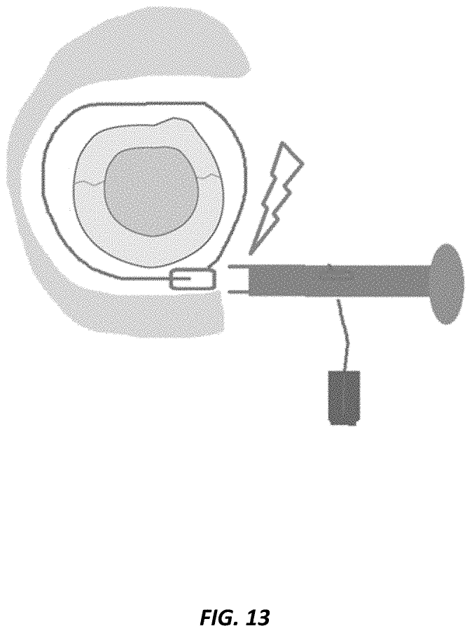

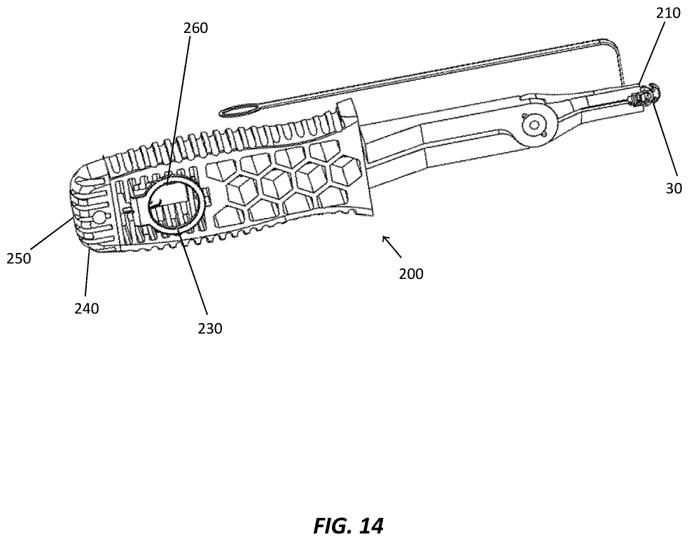

In an example, buckle 30 may include a pair of slots 35 located on opposite axial sides of buckle 30 relative to bone 20 as depicted in FIG. 1. Slots 35 may be configured (e.g., shaped and dimensioned) to receive arms 210 of a tensioner 200 depicted in FIG. 14 and depicted schematically in FIGS. 9-13. After cord 40 is passed around bone 20 and connected to buckle 30, proximal end 48 of cord 40 may be passed through a receiving passage 230 in tensioner 200 to allow cord 40 to be drawn or pulled by tensioner 200. Arms 210 may be received in slots 35 to connect tensioner 200 to buckle 30.

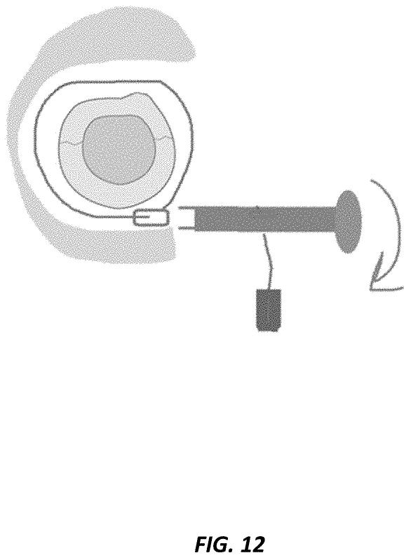

A handle 240 of tensioner 200 may be rotated by a user to cause the drawing of cord 40 by tensioner 200 away from bone 20 and toward a proximal end 250 of tensioner 200 as depicted in FIG. 12. A force provided on cord 40 by the drawing by tensioner 200 may pull cord 40 around lock rods 70 of buckle 30 to tighten cord 40 around bone 20, as described above, such that cord 40 is held at a particular tightness by friction provided by the path of cord 40 around lock bars 70 to allow a reduction of fracture 14, for example.

Tensioner 200 may include a rod 260 connected to handle 240 and including passage 230 to provide the drawing of cord 40 as described.

After cord 40 is tightened a desired amount around bone 20, e.g., when fracture 14 is placed in a desired position as determined by a user or surgeon, cord 40 may be cut such that proximal end 48 is at or adjacent to proximal end 31 of buckle 30 such as depicted in FIG. 1. Any sutures in the area around the fracture may be closed with buckle 30 and cord 40 remaining.

As indicated, buckle 30 and cord 40 may be utilized to for temporary fixation of a bone (e.g., fracture 14 of bone 20) with the buckle and cord being removed after a period of time (e.g., after the bone has healed) or buckle 30 and cord 40 may remain in vivo after the bone has healed. In an example, a bone plates may be placed over a tape (e.g., cord 40), to allow such a plate to be attached to appropriate portions of a bone (e.g., bone 20) cord 20 and buckle 30 are in place to reduce a fracture (e.g., fracture 14) Cord 40 may be cut and removed if the plate is adequate to hold the bone and cord 40 is not also needed. Such plates may also be applied to a bone other than over a cord to hold such bone to reduce a fracture, for example. Such plates or other surgical hardware may also remain in vivo with or without such a buckle and cord (e.g., buckle 30 and cord 40) hardware to stabilize a bone (e.g., a fracture 14) during a consolidation thereof.

In an example, multiple instances of a buckle and cord (e.g., buckle 30 and cord 40) may be utilized to fixate a fracture (e.g., fracture 14) or otherwise to hold a bone (e.g., bone 20) together at various longitudinal points along such a bone. A fracture may thus be segmentally reduced by incrementally drawing fragments of bone (e.g., bone 20) together. Such fracture reduction may be a dynamic operation and forces may need to be redirected due to an often-complex geometry of mating faces that may be adjusted and moved back together by manipulating a series of buckles and cord (e.g., multiple instances of buckle 30 and cord 40).

In another example not depicted, a holding member (not shown) may be used in place of tensioner 200 with the holding member including arms configured as arms 210 described above for tensioner 200, but not including a mechanism for drawing cord 40. Instead, the holding member may include a holding portion extending away from the arms to allow a user to hold the holding member thereby holding buckle 30 via the arms. While holding the holding member to hold the buckle a user may pull proximal end 48 of cord 40 to secure cord 40 around bone 20 to reduce fracture 14, for example.

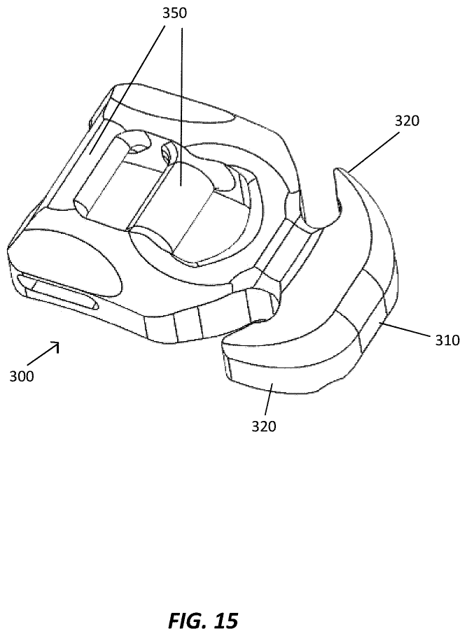

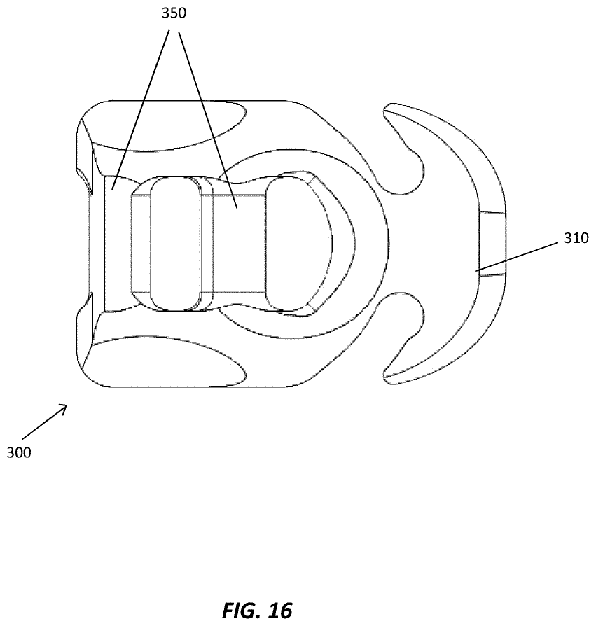

In another example depicted in FIGS. 15-16, a buckle 300 may be utilized in place of buckle 30 described above and may include a hook 310 having two outwardly facing prongs 320 configured to be attached to loop 44 of cord 40. Lock bars 350 may be utilized to connect to cord 40 as described above for lock bars 70.

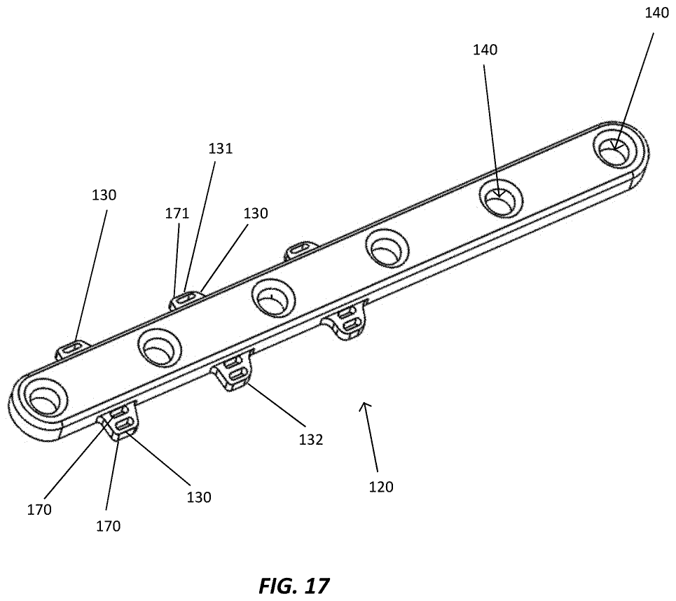

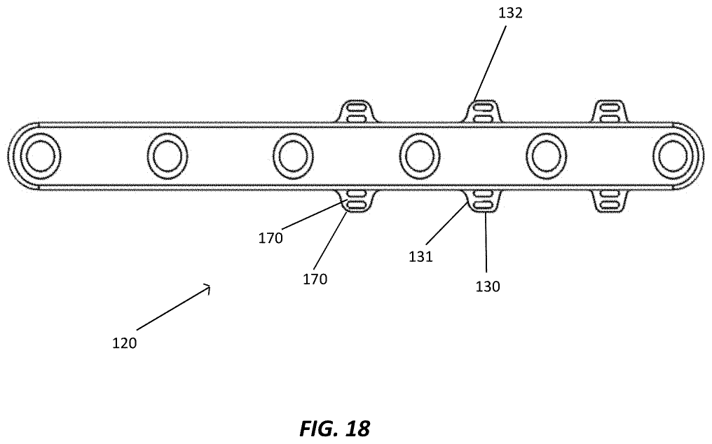

In an example depicted in FIGS. 17-18, a plate 120 may include buckles 130 having lock bars 170 and a plurality of openings 140 for receiving screws therethrough to allow plate 120 to be fastened to a bone (e.g., bone 20). A cord (e.g., cord 40) may be attached to a first buckle 131 and a second buckle 132 of buckles 130 as described above for cord 40 being attached to buckle 30. In an undepicted example, second buckle 132 may be replaced by a hook, similar to hook 50 described above, and a cord (e.g., cord 40) may be attached to lock bars 171 of buckle 131 and the hook as described for lock bars 70, hook 50 and cord 40. Buckles. Multiple instances of such buckles may be present along a length of a plate (e.g., plate 120). Each instance of a cord (e.g., cord 40) attached to opposing buckles (e.g.., first buckle 131 and second buckle 132) may be tensioned at a same or differing amount around a bone (e.g., bone 20) to achieve a particular therapeutic purpose for reducing a fracture (e.g. , fracture 14) Screws may be inserted though openings 140 after one or more such cords are attached to one or more of buckles 130 along plate 120 to secure and reduce a fracture in a bone (e.g., bone 20).

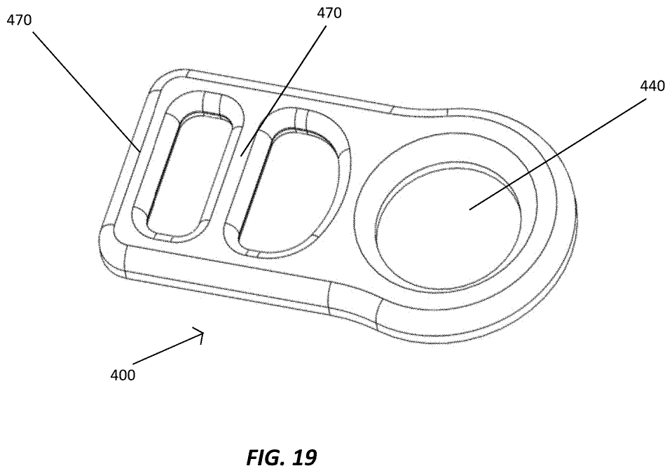

In an example depicted in FIG. 19, a washer 400 may include lock bars 470 and an opening 440 for receiving screws therethrough to washer 400 to be fastened to a bone (e.g., bone 20). Washer 400 may be located at a desired point on a bone (e.g., bone 20) where no other hardware is placed or washer 400 may be located on a plate such that opening 440 is aligned with a hole of the plate to allow a screw to pass through opening 440 and the opening of the plate to connect washer 400 to such plate. A cord (e.g., cord 40) may be attached to lock bars 470 and lock bars or a hook of another washer, buckle or plate to hold portions of a bone (e.g., bone 20) together to reduce a fracture, for example. In other examples, lock bars may be integrated into other fracture fixation hardware, e.g., washers, plates, toothed washers, and bone anchors. Buckles in these cases may have both ends of a cord (e.g., cord 40) tape secured or the cord may be secured between two discrete pieces of hardware (e.g., first buckle 131, second buckle 132, and washer 400).

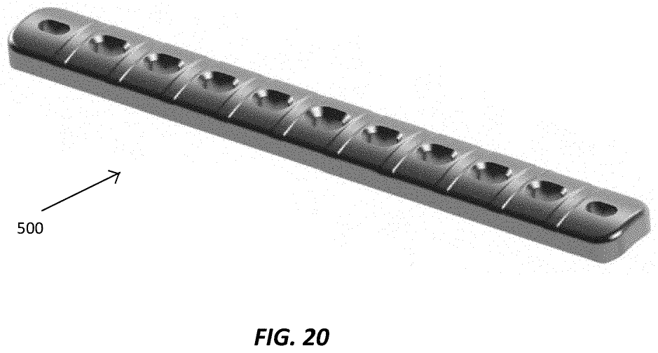

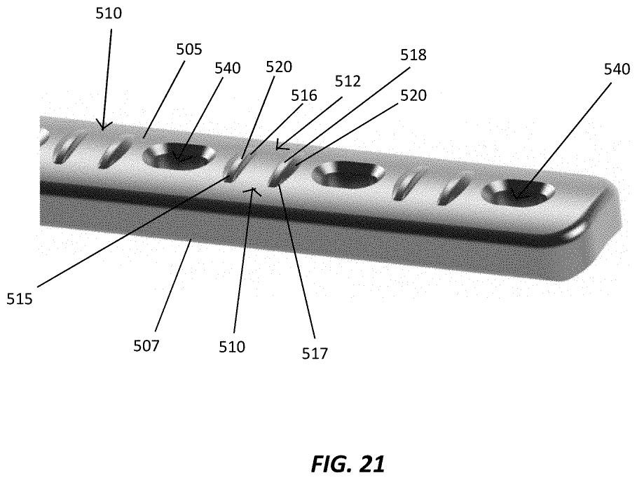

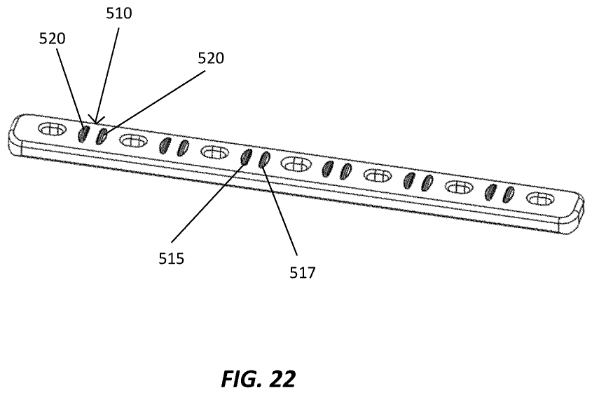

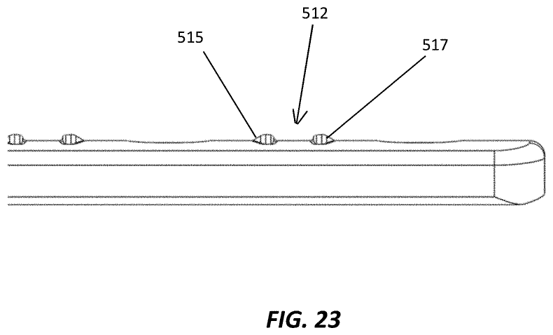

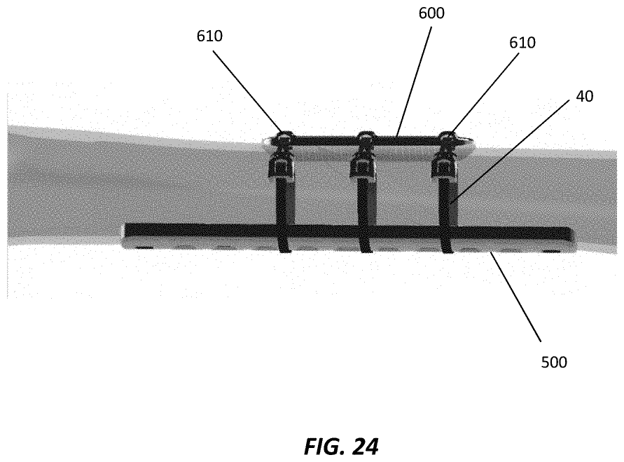

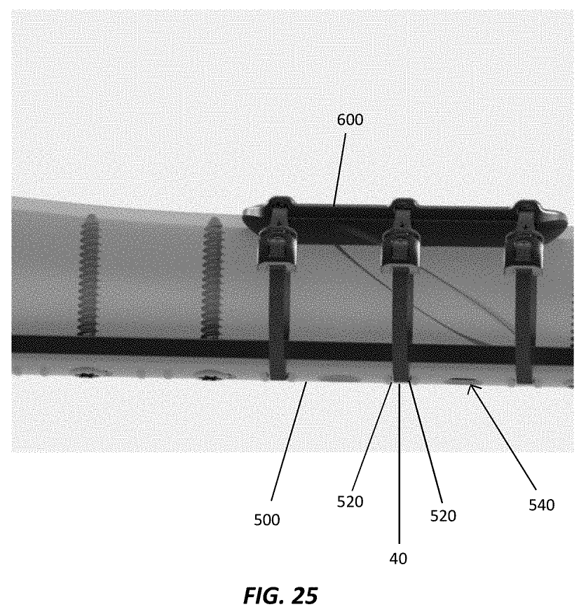

In another example depicted in FIGS. 20-23, a plate 500 is similar to plate 120 and may include a top side 505 opposite a bone contacting surface 507. A plurality of openings 540, similar to openings 140, may receive screws therethrough to allow plate 500 to be fastened to a bone (e.g., bone 20). Top side 505 may include a plurality of channels 510 bounded axially by anti-migration projections 520 on opposing axial sides thereof. For example, a first projection 515 and a second projection 517 of projections 520 may bound a channel 512 of channels 510. First projection 515 may have a first side 516 bounding channel 512 which is flat and extends about perpendicularly, relative to an axial dimension of plate 500, away from a bone contacting surface 507. Second projection 517 may have a second side 518 bounding channel 512 which is flat and extends about perpendicularly, relative to the axial dimension of plate 500, away from bone contacting surface 507. Channels 510 may be configured (e.g., axially shaped and dimensioned) to receive a cord or tape (e.g., cord 40) such that projections 520 (e.g., first projection 515 and second projection 517) inhibit or prevent movement of the cord or tape when received in channels 510 (e.g.., channel 512) as depicted for example in FIGS. 24-25.

FIG. 24 depicts plate 500 being connected to a plate 600, similar to three-wire cerclage adjunct device 200 depicted in FIG. 17 and disclosed in co-owned U.S. Ser. No. 16/910,328 (our file no. 3768.085A) incorporated herein by reference, via multiple instances of cord 40 and buckles (e.g., buckle 300) with the cords being received in channels 510. Plate 600 may be shaped similar to the device in the co-owned application except that openings 610 in plate 600 through the device may be configured (e.g., shaped and dimensioned) to receive a cord or tape (e.g., cord 40). FIG. 25 depicts a close up of FIG. 24 while further depicting screws being received in openings 540 to connect plate 500 to bone 20. The screws may be inserted though openings 540 after one or more cords (e.g., cords 40) are fed through openings 610 in plate 600 and around plate 500 (e.g., contacting top surface 505) through channels 510 to secure and reduce a fracture in a bone (e.g., bone 20).

In an example, cord 40 may be passed around bone 20 as described above using passer 100. Cord 40 may be fed through openings 610 in plate 600 on an accessible side of bone 20 closest to a surgeon or user. Cord 40 may then be manipulated or pulled to move plate 600 to an opposite side of bone 20 such that plate 600 may be in a position as depicted in FIGS. 24-25 with plate 500 being located on an opposite side of bone 20. As indicated above, cord 40 may be located in channels 510 and multiple instances of cord 40 may be secured via multiple instances of buckle 30 as described above.

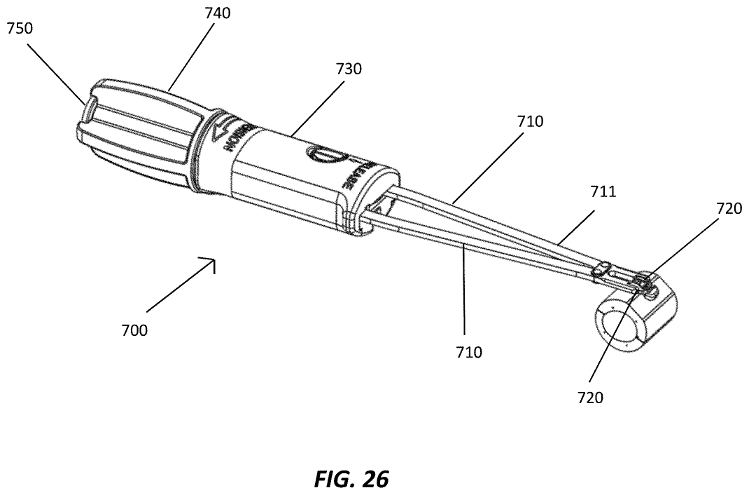

In an example, a tensioner 700 depicted in FIGS. 26-38 may include arms 710 with ends 720 configured (e.g., shaped and dimensioned) to receive a buckle (e.g., buckle 30 or buckle 300). For example, ends 720 may be configured to be received in slots 35 of buckle 30 as described above relative to tensioner 200. Arms 710 may be connected to a body 730 which may be connected to a rotatable tightener 740 configured to rotate relative to body 730 while attached to a cord (e.g., cord 40) to pull and provide tension to the cord while cord is around bone 20 as described above to facilitate the cord being secured around bone 20, plate 500 and plate 600, for example. A puller 750 located on an opposite end 752 of tensioner 700 relative to ends 720 may be connected to a cord (e.g., cord 40) after the cord is passed around bone 20, plate 500 and through plate 600 and puller may be pulled by a user to remove slack in cord 40 prior to tightener 740 being rotated to provide tension to cord to secure the cord and plates around bone 20 to fixate a fracture in bone 20, for example.

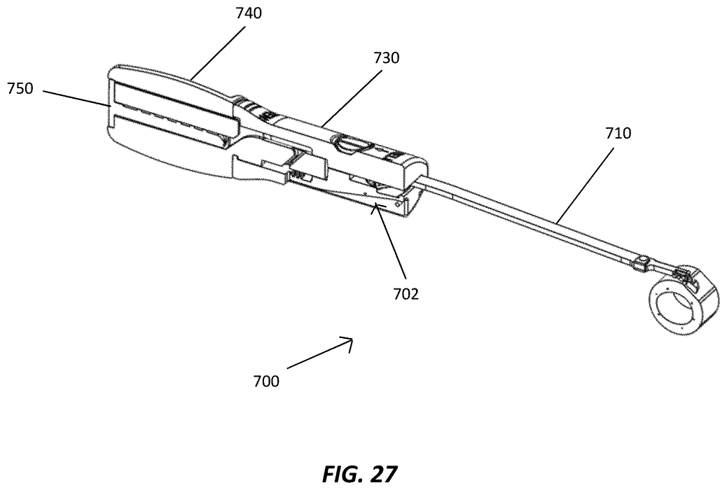

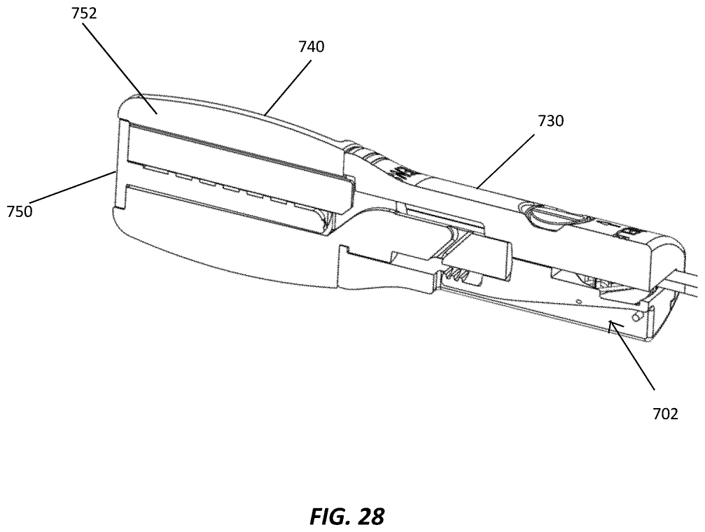

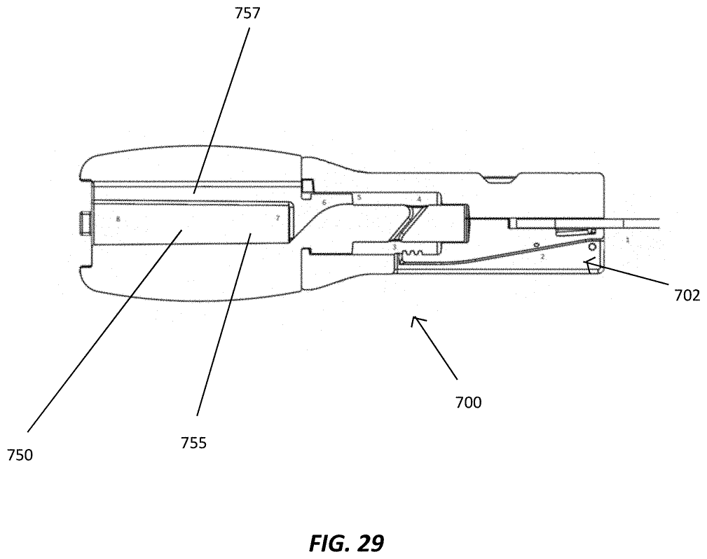

FIG. 27 depicts a cross-sectional view of FIG. 26 while FIG. 28 is a close up of a portion of FIG. 27. FIG. 29 is a side-sectional view of FIG. 26 further showing cord 40 extending through an interior 702 of tensioner 700. Cord 40 extends along a path outlined by a path portion 1 to a path portion 2 to a path portion 3 to a path portion 4 to a path portion 5 to a path portion 6 to a path portion 7 to a path portion 8 to a path portion 9 through the interior 702 of tensioner 700.

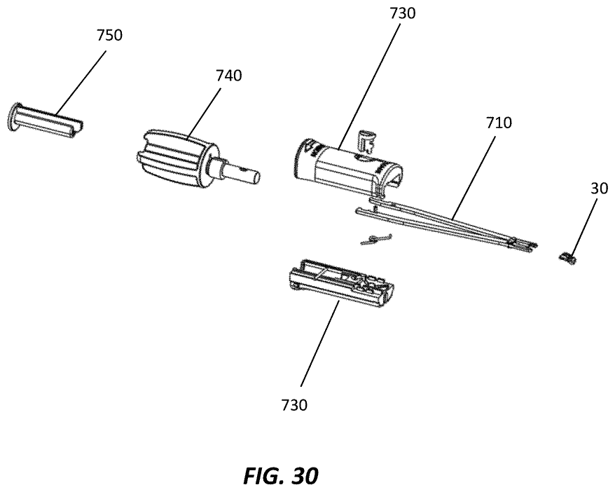

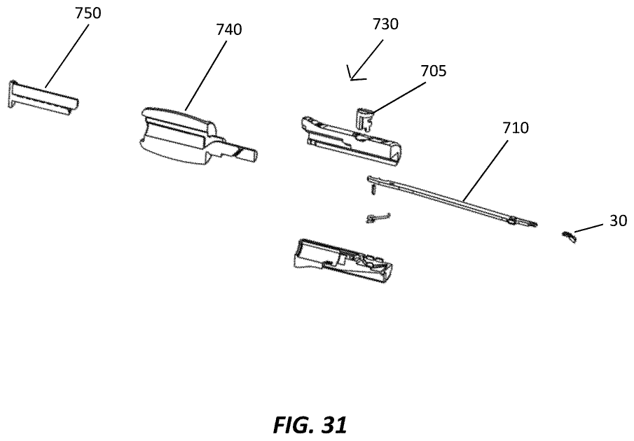

FIG. 30 is an exploded view of tensioner 700 while FIG. 31 is a cross-section of the tensioner of FIG. 30.

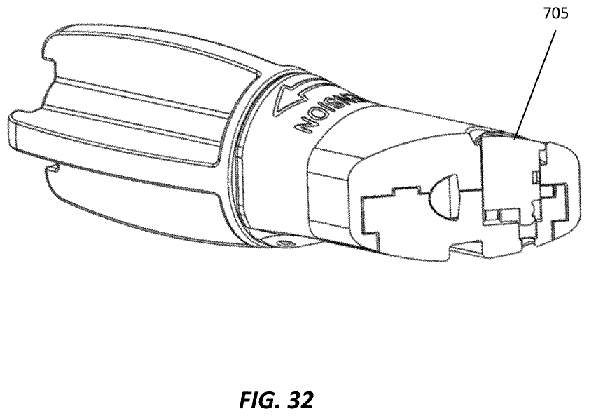

FIG. 32 depicts a cross-sectional view at an oblique angle relative to an axis of tensioner 700 showing a cut portion of body 730 depicting a position with a lock 705 in a release-locked position such that arms 710 are locked in a position holding a buckle (e.g., buckle 30). For example, the lock may maintain arms 710 in a particular position by inhibiting movement of the arms (e.g., proximal ends thereof) so that the arms (e.g., distal ends thereof) hold the buckle. In particular lock 705 may have a release slot 712 which is not aligned with an arm 711 of arms 710 in the locked position depicted in FIG. 32 thereby inhibiting movement of arms (e.g., proximal ends thereof) toward each to prevent release of the buckle.

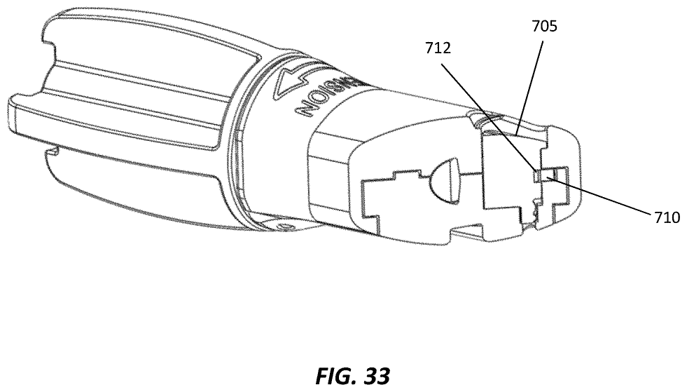

FIG. 33 depicts the tensioner in FIG. 32 with lock 705 depressed toward the axis of tensioner 700, allowing arms 710 (e.g., proximal ends thereof) to move freely towards each other (and toward the axis of tensioner 700) to release a buckle held therein. In an example, a movement of lock 705 to the unlocked position depicted in FIG. 33 allows movement of arm 711 into release slot 712 inwardly toward the axis of tensioner 700 such that an opposite end of one or more of arms 710 (e.g., distal ends thereof) may move outwardly away from axis of tensioner 700 to allow a release of the buckle (e.g., buckle 30).

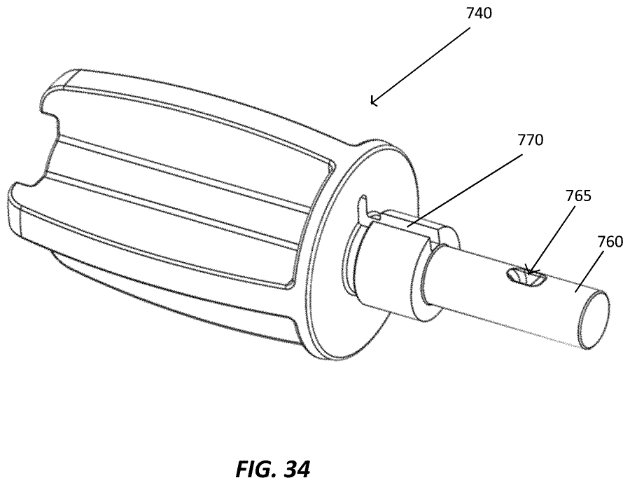

FIG. 34 depicts rotatable tightener 740 separate from a remainder of tensioner 700 and includes a rod portion 760 having a rod opening 765 to allow cord 40 to pass from path portion 3 to path portion 4 as described above. A path channel 770 allows cord 40 to pass from path portion 4 to path portion 6.

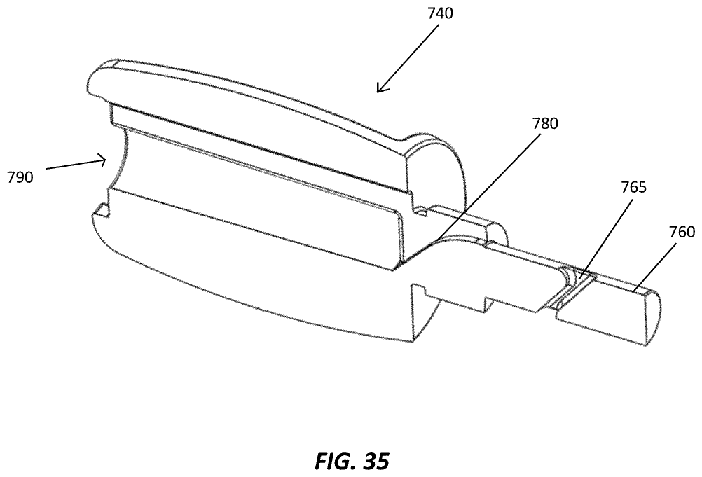

FIG. 35 is a cross-section of FIG. 34 depicting opening 765 aligned non-perpendicularly relative to the axis of tensioner 700 such that a bottom end as depicted is closer to tightener 740 than a top end thereof. As shown, a second path channel 780 may extend from path channel 770 in an arc toward and past the axis of tensioner 700 and extending to a cavity 790 for receiving puller 750. Cord 40 may be received in cavity 790 after passing thorough the interior of tensioner 700 as described above and may be connected to puller 750 such that the surgeon or user may reduce or eliminate any slack in cord 40 prior to a rotation of tightener 740 to place tension on cord 40 and securing buckle 30 to cord 40 as described above.

Tightener 740 may provide tension on cord 40 due to the cord being received in opening 765 such that the rotation of tightener 740 causes cord 40 to be wrapped around rod 760 thereby pulling on cord 40 in a direction away from arms 710 toward puller 750. Such tension provided to cord 40 may allow a buckle (e.g., buckle 30).

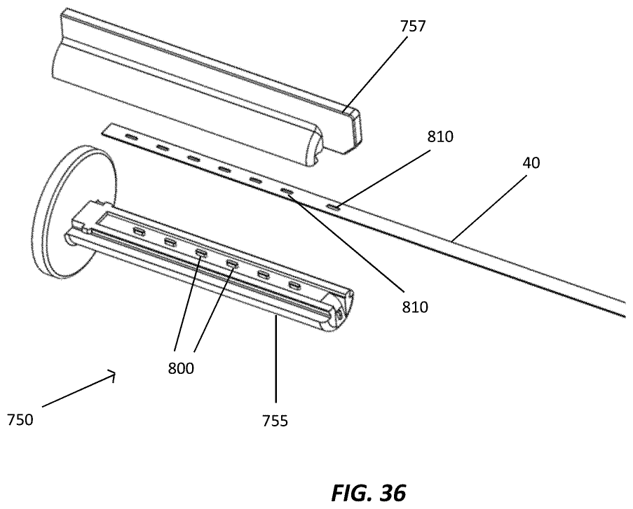

FIG. 36 depicts puller 750 in an exploded view including puller body 755 and puller cap 757 with cord 40 therebetween. Puller body 755 may include connecting projections 800 configured (e.g., shaped and dimensioned) to be received in openings 810 of cord 40 to connect puller 750 to cord 40. Puller cap 757 may abut or be connected to puller body 755 to inhibit movement of cord 40 away from puller body 744 when connecting projections 800 are received in openings 810. Cord 40 may be fed through interior of tensioner 700 as described above. Further, cord may be fed between puller body 755 and puller cap 757 followed by the cap being connected to, or abutting puller body 755 when puller 750 is received in cavity 790 and/or when puller 750 is pulled out of cavity 790 when attached to cord 40 to provide a force to cord 40 to eliminate slack in cord 40 and/or to provide a tension to cord 40 to allow a buckle (e.g., buckle 30) to be secured to cord 40.

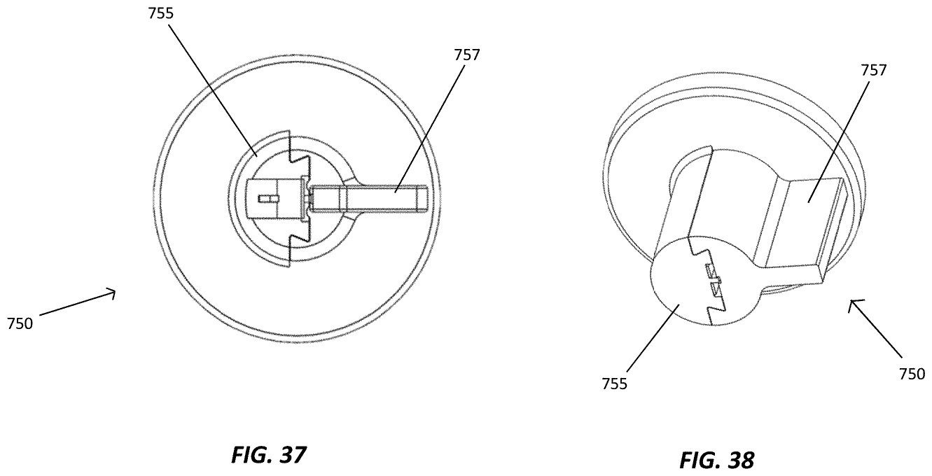

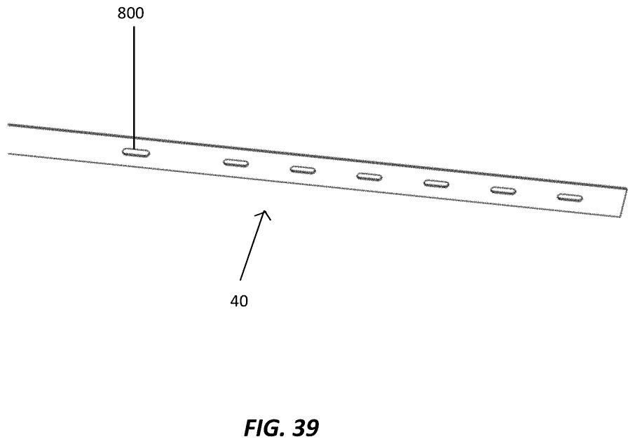

FIGS. 37 and 38 depict an end and perspective end view of puller body 755 and puller cap 757 engaged with cord 40 omitted from being shown therebetween.

FIG. 38 depicts a portion of cord 40 having laser cut holes for non-knotted termination (e.g., holes 800) as described above, where connecting projections 800 are configured (e.g., shaped and dimensioned) to be received in openings 810 of cord 40.

In an undepicted example, puller cap 757 could include projections which may extend through a cord (e.g., cord 40) and into cavities into puller body 755 to hold the cord instead of the reverse described above. Further puller body 755 may engage puller cap 757 without projections on either puller body 755 or puller cap 757, via other connecting mechanisms, or merely via friction, to hold a cord therebetween when connected.

In an example, tensioner 700 may be used in a method for fixating a bone (e.g., bone 20). As described above, cord 40 may be passed around a bone (e.g., bone 20) as described above using passer 100. Cord 40 may be fed through openings 610 in plate 600 on an accessible side of bone 20 closest to a surgeon or user and around plate 500 (e.g., through channels 510). Cord may be connected to buckle 30 and arms 710 of tensioner 700 may be received in slots of buckle 30. Lock 705 may be in a locked position as depicted in FIG. 32 such that arms 710 are locked to hold buckle 30.

Proximal end 48 of cord 40 may be passed through interior 702 through rod opening 765 and into cavity 790 to connect to puller 750 as described above. The user or surgeon may pull on puller 750 to minimize slack in cord 40. Tightener 740 may be rotated to a put cord 40 connected to buckle 30 at a desired tension such that the buckle may hold the cord to allow a reduction of fracture 14, for example.

Although a particular path through tensioner 700 is described above, such a tensioner could include any arrangement where a path for a cord (e.g., cord 70) extends through a tensioner to allow the cord to be pulled (e.g., via pulling by a person) to minimize slack and rotated (e.g., via a rotatable rod having an opening to receive the cord) to provide tension on a bone, the cord and any plates used to fixate a fracture. Further, although tensioner 700 is described as connected to buckle 30, the tensioner could be utilized with buckle 300, or any buckle configured for use in the fixation method described and having slots configured for engaging arms 710.

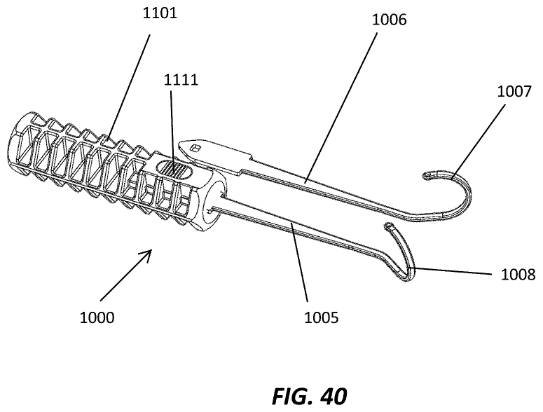

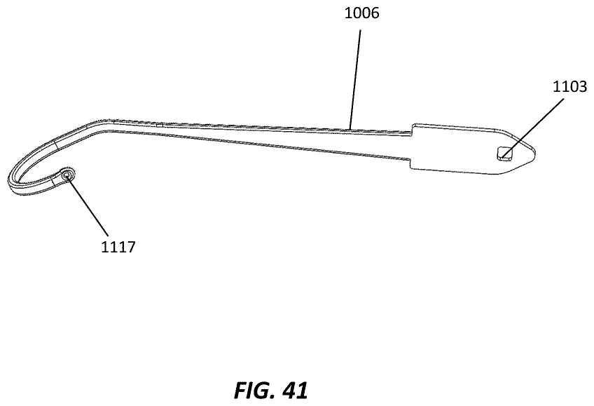

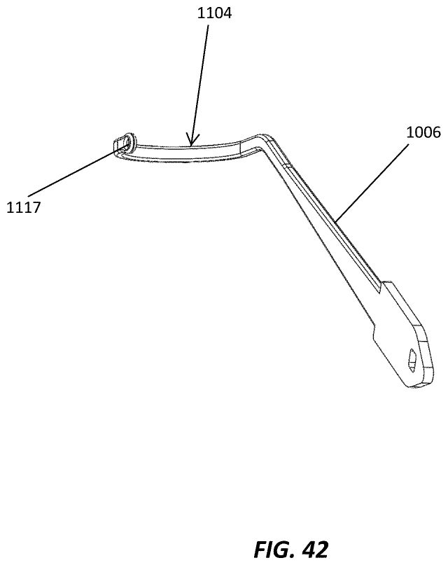

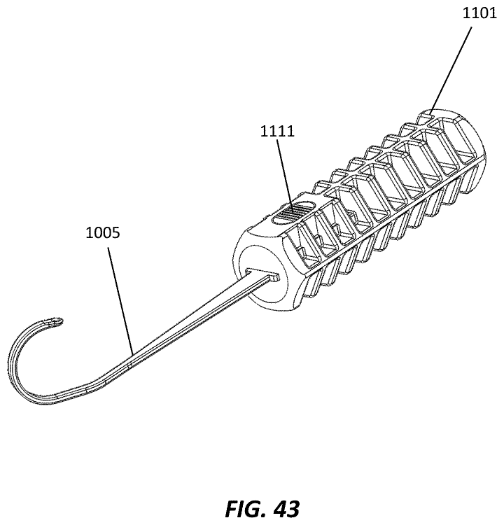

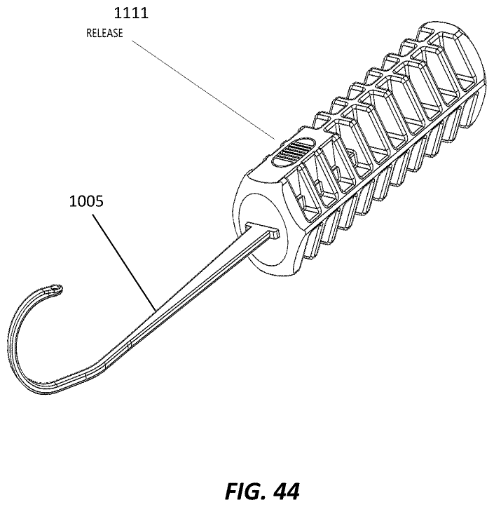

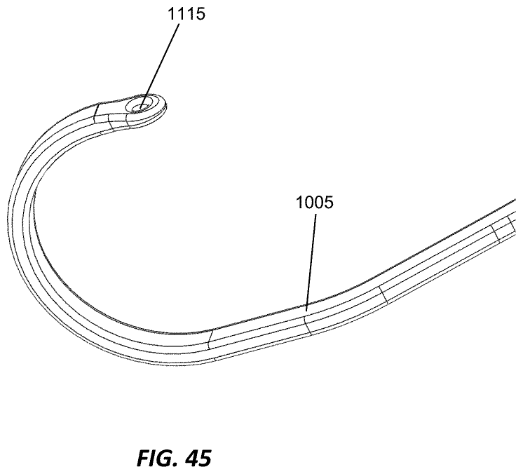

In another example depicted in FIGS. 40-46, a modular passer 1000 may be used in place of passer 100 (FIGS. 5-6) described above to pass a cord (e.g., cord 40) around a bone (e.g., bone 20) and under tissue and muscle (e.g., muscle 11). Passer 1000 may include a handle 1101 releasably connectable to a detachable arm 1005 having a curved extension portion 1008 with an inner circumferential side for receiving a cord (e.g., cord 40) prior to a procedure. As depicted in FIG. 40, a second arm 1006 may also be releasably connectable to handle portion 1101 having a second curved extension portion 1007.

As depicted in FIG. 40, for example, arm 1005 attached to handle 1101 may extend distally away from handle 1101 laterally opposite to a distal end of arm 1006 such that the arms (i.e., arm 1005 and arm 1006) may be utilized to allow a cord to passed around bones shaped and/or dimensioned differently from each other. Although not depicted, arms having different shapes and dimensions may also be connected to handle 1101 to substitute for arm 1005 and arm 1006 when passing a cord around a bone(s) having different arrangements.

Handle 1101 may include an engaging portion or projecting portion (not shown) configured to be received in a cavity (not shown) of arm 1005 when handle 1101 and arm 1005 are engaged and a locking switch 1111 is in a locked position such that the projecting portion is received in the cavity. Arm 1006 may also include a cavity 1103 to allow the projecting portion to be received in cavity 1103 to connect and lock arm 1006 relative to handle 1101. Locking switch may retract from the openings (e.g., cavity 1103) when in an unlocked position.

Passer 1000 may hold such a cord (e.g., cord 40) along an arm (e.g., arm 1005 or arm 1006) to manipulate the cord around a bone (e.g., bone 20) and under tissue and muscle, such that a distal end (e.g., distal end 46) thereof may be passed to an opposite side of the bone 20 relative to handle 1101 of passer 1000. Arm 1005 may include an opening 1115 and arm 1006 may include an opening 1117 to receive and hold a cord (e.g., cord 40) to hold the cord to allow the cord to be fed around the bone when arm 1005 and arm 1006 are, respectively, connected to handle portion 1101.

Passer 1000 may be removed from the bone (e.g., bone 20) with the cord (e.g., cord 40) remaining around the bone (e.g., bone 20) such that a distal end (e.g., distal end 46) and a proximal end (e.g., proximal end 48) may be located on a same side of bone 20.

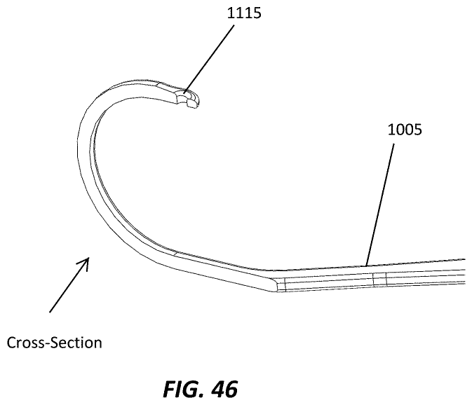

FIG. 46 depicts a cross-section of arm 1005. The cord (e.g., cord 40) may be removed from an opening (e.g., opening 1115 or opening 1117) of arm (e.g., arm 1005 or arm 1006) connected to handle 1101 after the cord has been passed around the bone to allow the cord to remain around the bone.

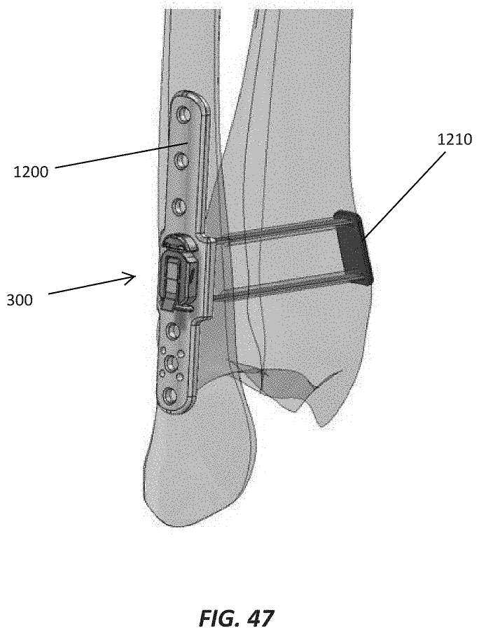

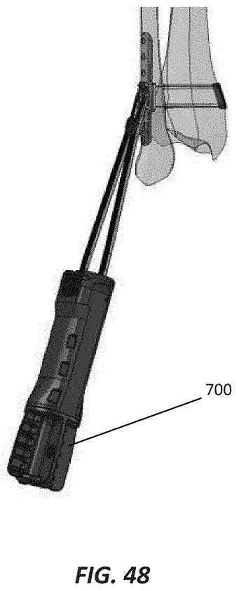

In an example depicted in FIG. 47, a buckle (e.g., buckle 300) may be utilized on a dorsal side of a fibular plate 1200 similar to plate 500 (FIG. 24) with the buckle located on a top side of the plate and a cord (e.g., cord 40) extending through openings in the plate around a bone to a second plate 1210. FIG. 48 depicts a tensioner (e.g., tensioner 700) being utilized to tighten the cord.

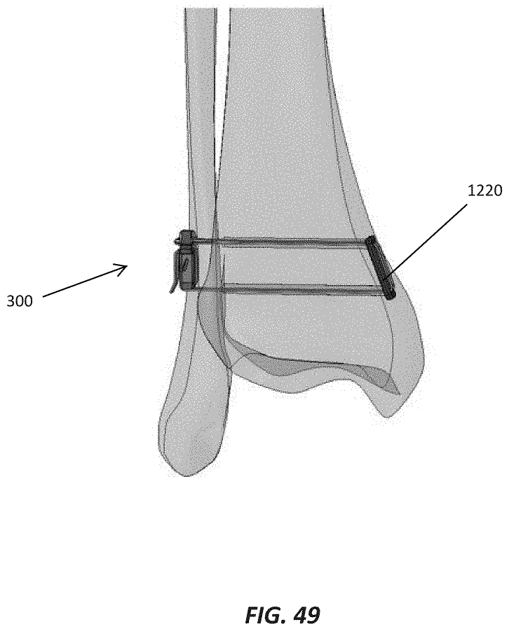

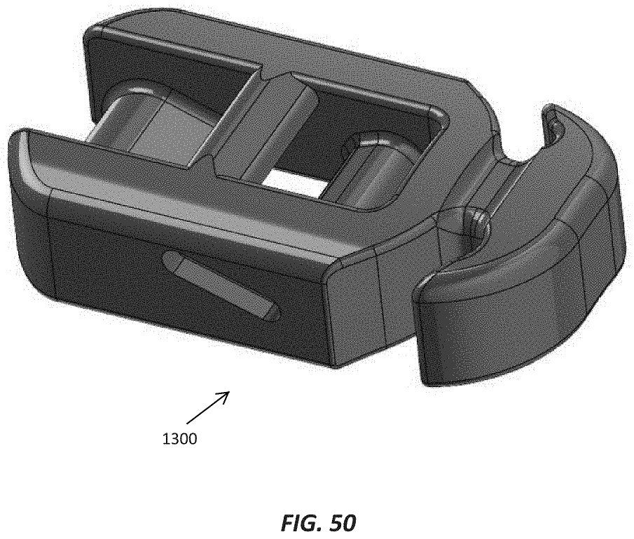

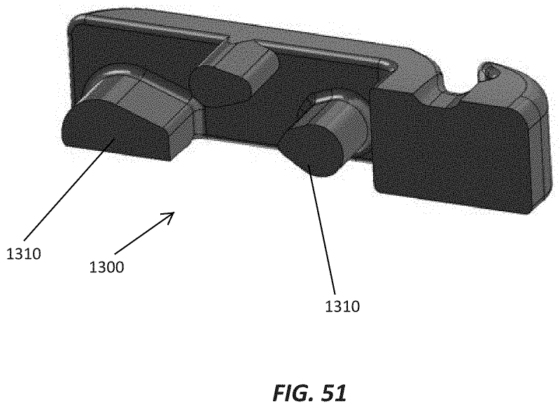

FIG. 49 depicts a buckle (e.g., buckle 300) attached and aligned as in FIG. 47 but without pate 1200 being utilized. FIG. 50 depicts another example of a buckle 1300 that may be utilized in a manner as the buckles (e.g., buckle 300) described above and FIG. 51 depicts a side cross-sectional view of the buckle of FIG. 50.

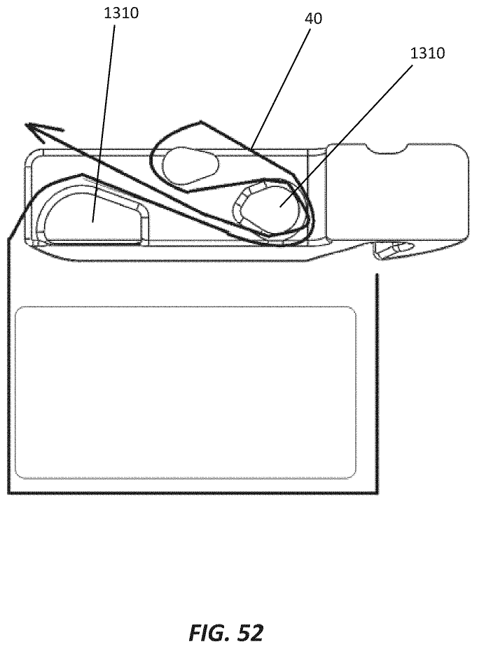

FIG. 52 depicts a path of a cord (e.g., cord 40) through bars 1310 of buckle 1300 similar to that of through bars 70 of buckle 30 described above and depicted in FIG. 2. to create a locking action on the cord.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

We claim:

1. A fracture fixation system comprising:
a connector having a proximal connecting end for connecting to a first end of a cord configured to extend around a bone to provide fixation to a fracture of the bone;
said connector comprising a distal connecting end opposite said connector relative to said proximal end for attaching to a second end of said cord extending around the bone; and
said proximal connecting end comprising a proximal lock member, a distal lock member and a medial lock member between said proximal lock member and said distal lock member, said proximal lock member located at a bottom of said proximal connecting end and said medial lock member located at a top of said proximal connecting end.

2. The system of claim 1 wherein said distal lock member is spaced from said bottom of said proximal connecting end.

3. The system of claim 1 wherein said distal connecting end comprises a hook or loop.

4. The system of claim 1 wherein said connector further comprises slots on opposite longitudinal sides of said proximal connecting end and aligned at an angle relative to a longitudinal axis of said connector, said slots configured to engage arms of a tensioner.

5. The system of claim 4 further comprising the tensioner engaged with said slots to hold said connector to allow a user to provide tension to the cord to fixate the fracture.

6. The system of claim 1 wherein said medial lock member comprises a cross-sectional tear drop shape having a smaller distal end than a proximal end thereof.

7. The system of claim 1 wherein said proximal lock member comprises a flat bottom side at said bottom of said proximal connecting end.

8. The system of claim 1 wherein said distal lock member is spaced from said bottom of said proximal connecting end and has a smaller proximal end than a distal end thereof

9. The system of claim 1 further comprising a cord contacting a top of said proximal lock member, extending around said distal lock member to said medial lock member, around said medial lock member to return to said distal lock member such that said cord is double layered on said distal lock member, and said cord extending from said distal lock member proximally away from said distal connecting end and exiting said proximal connecting end.

10. The system of claim 1 further comprising a first bone plate having a plurality of openings for receiving screws to connect said plate to the bone, said connector located on a top side of said plate and a cord extending through openings in the first bone plate, and around a bone to a second bone plate.

11. A method for fixating a bone comprising:
aligning opposing portions of a fractured bone to set the bone;
attaching a first end of a cord to a distal connecting end of a connector;
attaching a second end of the cord to a proximal connecting end of the connector by threading the cord through a plurality of slots bounded by a proximal lock member, a distal lock member and a medial lock member between the proximal lock member and the distal lock member, the proximal lock member located at a bottom of the proximal connecting end and the medial lock member located at a top of the proximal connecting end;
applying a force to the pull the cord around and through the proximal lock member, the distal lock member and the medial lock member lock member such that the cord is held at a particular tension to fixate the bone.

12. The method of claim 11 wherein the attaching the first end comprises attaching the first end to a hook or loop of the distal end.

13. The method of claim 11 further comprising engaging arms of a tensioner with slots of the connector to hold the connector, the slots located on opposite longitudinal sides of the connector and aligned at an angle relative to a longitudinal axis of the connector.

14. The method of claim 11 wherein the medial lock member comprises a cross-sectional tear drop shape having a smaller distal end than a proximal end thereof.

15. The method of claim 11 wherein the proximal lock member comprises a flat bottom side at said bottom of said proximal connecting end.

16. The method of claim 11 wherein the distal lock member is spaced from said bottom of said proximal connecting end and has a smaller proximal end than a distal end thereof

17. The method of claim 11 further comprising a cord contacting a top of the proximal lock member, extending around the distal lock member to the medial lock member, around the medial lock member to return to the distal lock member such that the cord is double layered on the distal lock member, and the cord extending from the distal lock member proximally away from the distal connecting end and exiting the proximal connecting end.

18. The method of claim 11 further comprising locating the connector on a first bone plate and the cord extending from the distal connecting end through openings in the first bone plate, around a bone through openings in a second bone plate and to the proximal connecting end.
